(19) United States
(12) Patent Application Publication
(10) Pub. No.: US 2009/0099486 A1
(43) Pub. Date: Apr. 16, 2009

(54) Ultrasonically Gas-Charged Reaction Accelerator

(76) Inventor: Mahesh C. Bhardwaj, State College, PA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 12/252,604

(22) Filed: Oct. 16, 2008

Related U.S. Application Data
(60) Provisional application No. 60/980,286, filed on Oct. 16, 2007.

(51) Int. Cl. A61N 7/00

(52) U.S. Cl. 601/2

(57) ABSTRACT

An apparatus comprises an ultrasound intensifier having an air/gas inlet, and a high powered non-contact ultrasound transducer, the ultrasound transducer directing ultrasound energy into the intensifier and methods of using the apparatus to increase the physical and/or chemical reactivity of a liquid or surface.

### BACKGROUND OF THE INVENTION

1. Field of the Invention

This application relates to the use of ultrasound to increase physical and/or chemical reactivity.

2. Description of Related Art

Non-contact ultrasonic transducers, particularly the type described in my U.S. Pat. No. 6,311,573, are characterized by extremely high transduction efficiency in air or other gaseous media. When excited by a suitable electrical energy source, such as by a pulsed or continuous wave power amplifier, non-contact ultrasound transducers create immense acoustic pressure, also supplemented by heat generation. Intense acoustic pressure zones exist in axial and transverse regions of ultrasonic fields in air/gas medium in which ultrasound is transmitted. In these regions, it is believed that intense heat is also generated due to air/gas molecular friction.

Evidence of these phenomena can be felt if one puts ones hand or any other part of the body one feels unusual sensation. Specifically, one gets a sensation that is not experienced in ordinary life events, such as those created by heat, pressure, or by chemical phenomena, for example, the feeling as if several needles are pounding the human skin and tissue, simultaneously generating unusual sensation of heat and pressure.

Similarly, if the surface of water or even solid material is exposed to such a field of ultrasound transmitted first through air, then one observes intense vibration of water or the material surface and its bulk. By raising or lowering the transducer in air with respect to its distance from the surface being irradiated by ultrasound, one observes maximum and minimum acoustic pressure zones (that can be observed with an unaided eye) which are separated by wavelength of ultrasound in air produced by the frequency of a given transducer.

By virtue of these observations, we decided to conduct an experiment to see if bacterial spores could be destroyed by using non-contact transducers. This experiment was successful and replicated several times and described in detail in a paper by Kelli Hoover, Mahesh Bhardwaj and Nancy Ostiguy entitled “Destruction of Bacterial Spores by High Power Non-Contact Ultrasound,” Mat. Res. Innovat., 6:291-295 (2002).

We have seen the observations described above by using non-contacting air or gas coupled transducers ranging in frequencies from 50 kHz to 500 kHz with active area diameters from 25 mm to 50 mm.

### SUMMARY OF THE INVENTION

Briefly, according to one embodiment of this invention, there is provided an apparatus comprising an ultrasound intensifier having an air/gas inlet, and a high-powered, non-contact ultrasound transducer, the ultrasound transducer directing ultrasound energy into the intensifier. Preferably, the ultrasound transducer is tuned to 50 to 500 kHz. Preferably, the apparatus has a conical shell narrowing to a tubular conduit and the ultrasound transducer is positioned at the wide end of the intensifier.

Briefly, according to other embodiments of this invention, there are provided methods of altering the physical or chemical reactivity of a liquid using the apparatus above described to direct ultrasound onto the surface of a liquid, to direct ultrasound below the surface of the liquid or to direct ultrasound onto the surface of a wound.

### BRIEF DESCRIPTION OF THE DRAWINGS

FIG. 1 is a schematic drawing illustrating the apparatus according to this invention for intensifying the surface of a liquid wherein the apparatus is spaced from the surface;

FIG. 2 is a schematic drawing illustrating the apparatus according to this invention for intensifying a liquid wherein the apparatus penetrates the liquid; and

FIG. 3 is a schematic drawing illustrating the apparatus according to this invention for treating a surface wound.

### DESCRIPTION OF THE PREFERRED EMBODIMENTS

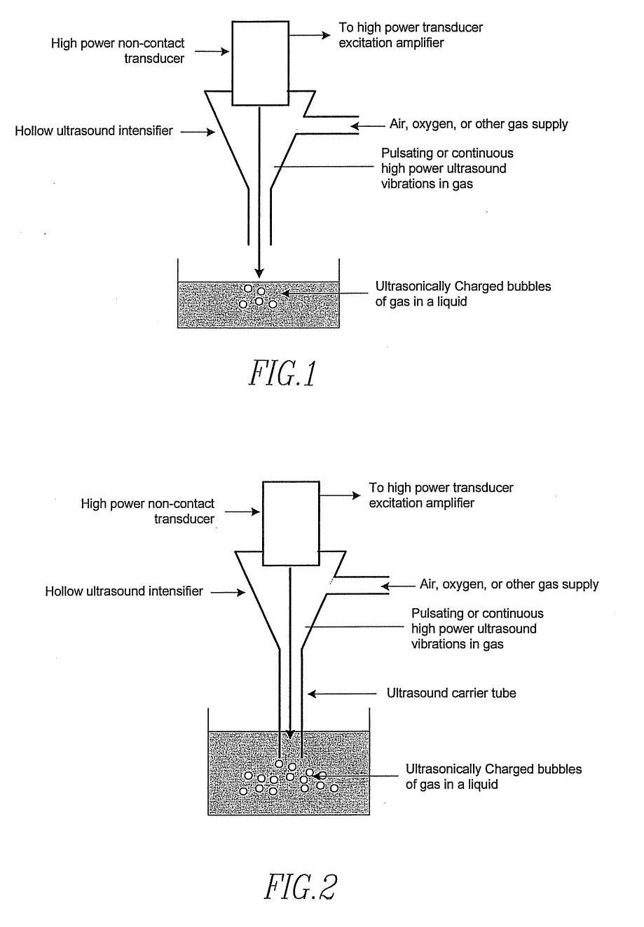

In the current invention, we envision application of high power ultrasound-charged air or other gases when it impinges on a liquid surface or on a soft surface, such as the human skin with wounds or other conditions, or on teeth and gums; or when ultrasonically-charged air/gas is inserted in a liquid, then it will alter the chemical and/or physical condition of the medium to which gas-charged ultrasound is applied. FIG. 1 illustrates the key components according to one embodiment of this invention.

Here, a non-contact, air or gas coupled transducer is excited by a suitable high power amplifier. The transducer is connected to a conical hollow ultrasound intensifier emitting high power ultrasound in gas.

When ultrasonically charged gas impacts a liquid surface, it breaks the gas-liquid boundary and penetrates inside the liquid, thus inflicting change in the liquid.

FIG. 2 illustrates the key components according to another embodiment of this invention. Here, a non-contact air or gas coupled transducer is excited by a suitable high power amplifier. The transducer is connected to a conical hollow ultrasound intensifier emitting high power ultrasound in gas that is extended by an ultrasound carrier tube.

When ultrasonically-charged gas is released in the liquid it reacts with the liquid, thus inflicting change in it.

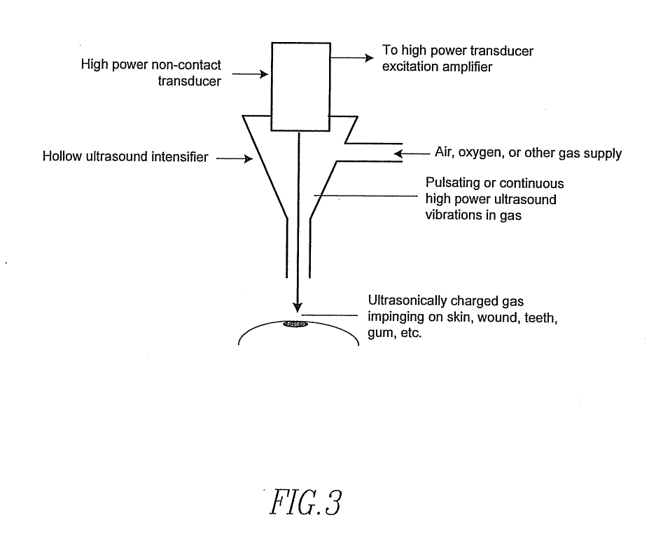

FIG. 3 illustrates the key components according to yet another embodiment of this invention. Here, a non-contact air or gas coupled transducer is excited by a suitable high power amplifier. The transducer is connected to a conical hollow ultrasound intensifier.

When ultrasonically-charged gas hits skin or a wound, or teeth, gums, etc., their characteristics are altered, thereby setting the stage for healing the wound or changing the skin condition, and healing gums or cleaning teeth, etc.

Having thus described my invention with the detail and particularity required by the Patent Laws, what is desired protected by Letters Patent is set forth in the following claims.

The invention claimed is:

1. An apparatus comprising:
an ultrasound intensifier having an air/gas inlet; and
a high powered non-contact ultrasound transducer, said ultrasound transducer directing ultrasound energy into the intensifier.

2. The apparatus according to claim 1, wherein the ultrasound transducer is tuned to 50 to 500 kHz.

3. The apparatus according to claim 1, wherein the ultrasound intensifier has a conical shell narrowing to a tubular conduit and the ultrasound transducer is positioned at the wide end of the intensifier.

4. A method of altering the physical or chemical reactivity of a liquid using the apparatus of claim 1 to direct ultrasound onto the surface of a liquid.

5. A method of altering the physical or chemical reactivity of a liquid using the apparatus of claim 1 to direct ultrasound below the surface of the liquid.

6. A method of altering the physical or chemical reactivity of a wound using the apparatus of claim 1 to direct ultrasound onto the surface of the wound.
